(19) United States
(12) Patent Application Publication
(10) Pub. No.: US 2017/0258749 A1
(43) Pub. Date: Sep. 14, 2017

(54) Oseltamivir Compositions

(71) Applicant: Lupin Atlantis Holdings SA, Zug (CH)

(72) Inventors: Ketan Bhasale, Pune (IN); Abhishek Shah, Pune (IN); Romesh Jha, Pune (IN); Subhasis Das, Pune (IN); Vijaya Kumar Thommandru, Pune (IN)

(21) Appl. No.: 15/531,881

(22) PCT Filed: Nov. 30, 2015
(86) PCT No.: PCT/IB2015/059198

§ 371 Date: May 31, 2017

(30) Foreign Application Priority Data
Dec. 1, 2014 (IN) 1262/KOL/2014

(51) Int. Cl. A61K 31/215; A61K 47/26; A61K 47/12; A61K 9/10

(52) U.S. Cl. CPC A61K 31/215; A61K 9/10; A61K 47/26; A61K 47/12

(57) ABSTRACT

The present invention relates to pharmaceutical composition comprising two different populations with first population comprising oseltamivir or a pharmaceutical acceptable salt thereof and one or more pharmaceutically acceptable excipients and second population comprising one or more pharmaceutically acceptable excipients. Preferably, the compositions wherein the second population does not contain oseltamivir or a pharmaceutically acceptable salt thereof. The invention also disclose new method of filing the composition into container. The inventors of the present invention surprisingly found that the composition are stable in real-time and long-term stability conditions. Further, the compositions are bioequivalent to marketed suspension formulation of Oseltamivir phosphate.

### FIELD OF INVENTION

The present invention relates to pharmaceutical compositions comprising Oseltamivir, preferably compositions for constitution into suspensions and process for preparation thereof.

### BACKGROUND OF INVENTION

U.S. Pat. No. 5,763,483 discloses oseltamivir as (3R,4R,5S)-4-acetylamino-5-amino3(1-ethylpropoxy)-1-cyclohexene-1-carboxylic acid, ethyl ester, phosphate and its pharmaceutical acceptable salts and their use in the treatment of influenza.

Oseltamivir is marketed as capsule and powder for oral suspension wherein oseltamivir is present in the form of Oseltamivir phosphate.

Oseltamivir compound has an amine group that can react with reducing sugars which may result in the discoloration of composition. In order to avoid this discoloration, U.S. patent application no. 20100222427 discloses a physicochemically stable powder for suspension of oseltamivir phosphate comprising sugar or sugar alcohols in which equilibrium water content is 1% by weight or less at 25° C. and 70% relative humidity, wherein each of glucose and mannose contained in the sugars and sugar alcohols as impurities is 0.01% by weight or less.

There continues to be a need to provide new compositions physicochemically stable powder for suspension containing oseltamivir phosphate.

### OBEJECTIVES OF INVENTION

The main object of the invention is to provide pharmaceutical compositions of oseltamivir or a pharmaceutical acceptable salt thereof and a process for preparation thereof. The pharmaceutical compositions of the invention are preferably compositions for constitution into suspension.

Another object of the invention is a pharmaceutical composition comprising two different populations: i) first population comprising oseltamivir or a pharmaceutical acceptable salt thereof and one or more pharmaceutically acceptable excipients and ii) second population comprising one or more pharmaceutically acceptable excipients.

Another object of the invention is a pharmaceutical composition comprising two different populations: i) first population is in the form of powder comprising oseltamivir or a pharmaceutical acceptable salt thereof and one or more pharmaceutically acceptable excipients and ii) second population is in the form of granules comprising one or more pharmaceutically acceptable excipients.

Another object of the invention is a pharmaceutical composition comprising two different populations: i) first population is in the form of granules comprising oseltamivir or a pharmaceutical acceptable salt thereof and one or more pharmaceutically acceptable excipients and ii) second population is in the form of granules comprising one or more pharmaceutically acceptable excipients.

Another object of the invention is a pharmaceutical composition comprising two different populations: i) first population is in the form of granules comprising oseltamivir or a pharmaceutical acceptable salt thereof and one or more pharmaceutically acceptable excipients and ii) second population is in the form of powder comprising one or more pharmaceutically acceptable excipients.

Another object of the invention is a pharmaceutical composition comprising two different populations, wherein the ratio of first population comprising oseltamivir or a pharmaceutical acceptable salt thereof to second population comprising excipients is in the range of 1:99 to 99:1 based on total weight of the composition.

Another object of the invention is a pharmaceutical composition comprising two different populations, wherein first population comprises oseltamivir or a pharmaceutically acceptable salt thereof in an amount of 5% to 80% by weight based on the total weight of first population.

Another object of the invention is a pharmaceutical composition comprising two different populations, wherein first population comprises oseltamivir or a pharmaceutically acceptable salt thereof in an amount of about 5% to about 80% by weight based on the total weight of first population.

Another object of the invention is a pharmaceutical composition comprising two different populations: i) first population comprising oseltamivir or a pharmaceutical acceptable salt thereof and one or more pharmaceutically acceptable excipients and ii) second population comprising one or more pharmaceutically acceptable excipients, wherein the second population does not contain oseltamivir or a pharmaceutical acceptable salt thereof.

Another object of the invention is a pharmaceutical composition comprising two different populations: i) first population is in the form of powder comprising oseltamivir or a pharmaceutical acceptable salt thereof and one or more pharmaceutically acceptable excipients and ii) second population is in the form of granules comprising one or more pharmaceutically acceptable excipients, wherein the second population does not contain oseltamivir or a pharmaceutical acceptable salt thereof.

Another object of the invention is a pharmaceutical composition comprising two different populations: i) first population comprising oseltamivir or a pharmaceutical acceptable salt thereof and one or more pharmaceutically acceptable excipients and ii) second population comprising one or more pharmaceutically acceptable excipients, wherein the second population does not contain oseltamivir or a pharmaceutical acceptable salt thereof, and wherein the composition is stable throughout its shelf life.

Another object of the invention is a pharmaceutical composition comprising two different populations: i) first population is in the form of powder comprising oseltamivir or a pharmaceutical acceptable salt thereof and one or more pharmaceutically acceptable excipients and ii) second population is in the form of granules comprising one or more pharmaceutically acceptable excipients, wherein the second population does not contain oseltamivir or a pharmaceutical acceptable salt thereof, and wherein the composition is stable throughout its shelf life.

Another object of the invention is a pharmaceutical composition comprising two different populations: i) first population is in the form of powder comprising oseltamivir or a pharmaceutically acceptable salt thereof and sugar alcohol and ii) second population is in the form of granules comprising sugar alcohol.

Another object of the invention is a process of preparing composition comprising:
i. mixing oseltamivir or a pharmaceutically acceptable salt thereof with one or more pharmaceutically acceptable excipients to form first population;
ii. forming granules of pharmaceutically acceptable excipients by using garnulation technique to form second population;
iii. filling first population of step (i) into the container with first nozzle;
iv. filling second population of step (ii) into the container of step (iii) by second nozzle.

### DETAILED DESCRIPTION OF THE INVENTION

Oseltamivir compound has an amine group that can react with reducing sugars which may result in the discoloration of composition. Numerous attempts were made in the past to avoid this discoloration by applying various conditions. These conditions and requiremements are difficult to achieve, time-consuming, complex and costly to get a stable composition of oseltamivir. However, surprisingly it was found that stable pharmaceutical compositions of oseltamivir can be prepared having two different populations. The present invention relates to a pharmaceutical compositions of oseltamivir and a process for preparation thereof. More particulalry, pharmaceutical compositions are in the form of compositions for constitution into suspension and process for preparation thereof.

Pharmaceutical compositions of invention have uniform distribution of active ingredient and are stable throughout shelf life. Further, pharamaceutical compositions of invention have comparable in-vitro dissolution profile with marketed suspension formulation of Oseltamivir phosphate. More preferably, pharmaceutical compositions of invention are bioequivalent to marketed suspension formulation of Oseltamivir phosphate.

“Oseltamivir” used in the present invention is in the form of base or pharmaceutically acceptable derivative like esters(s) or salt(s) or enantiomer(s) or polymorph(s) or solvates thereof. Preferably Oseltamivir is in the form of pharmaceutically acceptable salt form. More preferably, pharmaceutically acceptable salt is phosphate salt.

Pharmaceutical compositions of Oseltamivir or a pharmaceutically acceptable salt thereof according to the invention comprise but are not limited to suspensions, solutions, emulsions, ointments, liniments, lotions, creams, gels, suppositories, transdermal patches, powders and osmotic pumps, tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release formulations, capsules, microcapsules, tablets in capsules, microspheres, matrix formulations, microencapsulation.

More preferably, compositions of invention comprise powders, granules or mixture thereof for constitution into suspension. Most preferably, compositions of invention comprise mixture of granules and powder for constitution into suspension.

In one embodiment, a pharmaceutical composition comprising two different populations: i) first population comprising oseltamivir or a pharmaceutically acceptable salt thereof and one or more pharmaceutically acceptable excipients and ii) second population comprising one or more pharmaceutically acceptable excipients.

In another embodiment, a pharmaceutical composition comprising two different populations: i) first population is in the form of powder comprising oseltamivir or a pharmaceutically acceptable salt thereof and one or more pharmaceutically acceptable excipients and ii) second population is in the form of granules comprising one or more pharmaceutically acceptable excipients.

In another embodiment, a pharmaceutical composition comprising two different populations: i) first population is in the form of granules comprising oseltamivir or a pharmaceutically acceptable salt thereof and one or more pharmaceutically acceptable excipients and ii) second population is in the form of granules comprising one or more pharmaceutically acceptable excipients.

In another embodiment, a pharmaceutical composition comprising two different populations: i) first population is in the form of granules comprising oseltamivir or a pharmaceutically acceptable salt thereof and one or more pharmaceutically acceptable excipients and ii) second population is in the form of powder comprising one or more pharmaceutically acceptable excipients.

In another embodiment, a pharmaceutical composition comprising two different populations: i) first population is in the form of granules comprising oseltamivir or a pharmaceutically acceptable salt thereof and one or more pharmaceutically acceptable excipients and ii) second population is in the form of powder comprising one or more pharmaceutically acceptable excipients and wherein the composition is stable throughout its shelf life.

In another embodiment, a pharmaceutical composition comprising two different populations: i) first population is in the form of powder comprising oseltamivir or a pharmaceutically acceptable salt thereof and one or more pharmaceutically acceptable excipients ii) second population is in the form of granules comprising one or more pharmaceutically acceptable excipients, wherein the second population does not contain oseltamivir or a pharmaceutically acceptable salt thereof.

In another embodiment, a pharmaceutical composition comprising two different populations: i) first population is in the form of granules comprising oseltamivir or a pharmaceutically acceptable salt thereof and one or more pharmaceutically acceptable excipients ii) second population is in the form of granules comprising one or more pharmaceutically acceptable excipients, wherein the second population does not contain oseltamivir or a pharmaceutically acceptable salt thereof.

In another embodiment, a pharmaceutical composition comprising two different populations: i) first population is in the form of granules comprising oseltamivir or a pharmaceutically acceptable salt thereof and one or more pharmaceutically acceptable excipients ii) second population is in the form of powder comprising one or more pharmaceutically acceptable excipients, wherein the second population does not contain oseltamivir or a pharmaceutically acceptable salt thereof.

In another embodiment, a pharmaceutical composition comprising two different populations i) first population comprising oseltamivir or a pharmaceutically acceptable salt thereof and one or more pharmaceutically acceptable excipients and ii) second population comprising one or more pharmaceutically acceptable excipients, wherein the second population does not contain oseltamivir or a pharmaceutically acceptable salt thereof, and wherein the composition is stable throughout its shelf life.

In another embodiment, a pharmaceutical composition comprising two different populations i) first population is in the form of powder comprising oseltamivir or a pharmaceutically acceptable salt thereof and one or more pharmaceutically acceptable excipients and ii) second population is in the form of granules comprising one or more pharmaceutically acceptable excipients, wherein the second population does not contain oseltamivir or a pharmaceutically acceptable salt thereof, and wherein the composition is stable throughout its shelf life.

In another embodiment, a pharmaceutical composition comprising two different populations i) first population is in the form of granules comprising oseltamivir or a pharmaceutically acceptable salt thereof and one or more pharmaceutically acceptable excipients and ii) second population is in the form of granules comprising one or more pharmaceutically acceptable excipients, wherein the second population does not contain oseltamivir or a pharmaceutically acceptable salt thereof, and wherein the composition is stable throughout its shelf life.

In another embodiment, a pharmaceutical composition comprising two different populations i) first population is in the form of granules comprising oseltamivir or a pharmaceutically acceptable salt thereof and one or more pharmaceutically acceptable excipients and ii) second population is in the form of powder comprising one or more pharmaceutically acceptable excipients, wherein the second population does not contain oseltamivir or a pharmaceutically acceptable salt thereof, and wherein the composition is stable throughout its shelf life.

In another embodiment a pharmaceutical composition comprising two different populations i) first population is in the form of granules comprising oseltamivir or a pharmaceutically acceptable salt thereof and one or more pharmaceutically acceptable excipients and ii) second population is in the form of powder comprising one or more pharmaceutically acceptable excipients, wherein the second population does not contain oseltamivir or a pharmaceutically acceptable salt thereof, wherein the composition is stable while kept in real-time stability conditions as well as long-term stability conditions (30° C.±2° C./65% RH±5% RH).

The real-time stabiltiy condition means the stability studies carried out at temperature of 25° C.±2° C. and relative humidity (RH) of 60%±5%.

According to US Pharmacopoeia monograph for Oseltamivir Phosphate capsules, three impurities are identified to be associated with Oseltamivir, they are Impurity A, B and C.

In another embodiment a pharmaceutical composition comprising two different populations i) first population is in the form of granules comprising oseltamivir or a pharmaceutically acceptable salt thereof and one or more pharmaceutically acceptable excipients and ii) second population is in the form of powder comprising one or more pharmaceutically acceptable excipients, wherein the second population does not contain oseltamivir or a pharmaceutically acceptable salt thereof, wherein the composition has impurity A not more than 2.0% by weight as per the USP monograph for Oseltamivir Phosphate Capsules when kept in real-time and long-term stability conditions.

In another embodiment a pharmaceutical composition comprising two different populations i) first population is in the form of granules comprising oseltamivir or a pharmaceutically acceptable salt thereof and one or more pharmaceutically acceptable excipients and ii) second population is in the form of powder comprising one or more pharmaceutically acceptable excipients, wherein the second population does not contain oseltamivir or a pharmaceutically acceptable salt thereof, wherein the composition has impurity B not more than 0.3% by weight as per the USP monograph for Oseltamivir Phosphate Capsules when kept in real-time and long-term stability conditions.

In another embodiment a pharmaceutical composition comprising two different populations i) first population is in the form of granules comprising oseltamivir or a pharmaceutically acceptable salt thereof and one or more pharmaceutically acceptable excipients and ii) second population is in the form of powder comprising one or more pharmaceutically acceptable excipients, wherein the second population does not contain oseltamivir or a pharmaceutically acceptable salt thereof, wherein the composition has impurity C not more than 0.5% by weight as per the USP monograph for Oseltamivir Phosphate Capsules, when kept in real-time and long-term stability conditions.

In another embodiment pharmaceutical compositions of the invention has impurity A not more than 2.0% by weight, impurity B not more than 0.3% by weight and impurity C not more than 0.5% by weight as per the USP monograph for Oseltamivir Phosphate Capsules, while kept in real-time and long-term stability conditions.

In another embodiment, a pharmaceutical composition comprising two different populations, wherein the weight ratio of first population to second population is in the range of 1:99 to 99:1 based on the total weight of the composition. More preferably, the weight ratio of first population to second population is in the range of 40:60 to 5:95 based upon the total weight of the composition.

In another embodiment, a pharmaceutical composition comprising two different populations, wherein first population comprises oseltamivir or a pharmaceutically acceptable salt thereof in an amount of 5% to 80% by weight based on total weight of first population.

In another embodiment, pharmaceutical compositions of invention comprises granules, wherein granules can be prepared by using one or more techniques such as wet, dry granulation, direct compression, fluidized bed processor or any other techniques known on the art.

In another embodiment granules can be prepared by wet granulation, wherein granulation liquid can be aqueous, non-aqueous or hydroalcoholic.

In another embodiment, pharmaceutical compositions of invention are in the form of compositions for constitution into suspensions. Compositions of invention can be constituted into suspension by using one or more vehicles comprise but not limited to water, orange or lime juices, non aqueous liquids or mixtures thereof. Most preferably, compositions of invention are constituted into suspension using water as a vehicle.

The amount of Oseltamivir or a pharmaceutically acceptable salt form in pharmaceutical compositions of invention will be suitable amount as known in the art. Preferebly, Oseltamivir base is present in suspension after constitution using vehicles in the concentration (w/v) in between 1 mg/mL to 100 mg/mL, more preferably less than 25 mg/mL, in most preferably 6 mg/mL.

In another embodiment, suspensions after constitution according to invention can be administered to humans by oral administration.

The term ‘pharmaceutically acceptable excipient(s)’ used in the pharmaceutical compositions of invention comprise but not limited to diluents, binders, pH stabilizing agents, disintegrants, surfactants, glidants, lubricants, suspending agents, flavouring agents, sweetening agents, buffers, coloring agents or preservatives.

The amount of excipient(s) employed will depend upon how much active agent is to be used. One excipient(s) can perform more than one function.

Binders as used in the present invention comprises but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof. If possible, give ranges (including fallback ranges) for amount of binders present in the composition.

Fillers or diluents as used in the present invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the present invention comprises but not limited to magnesium stearate, polyethylene glycol, glyceryl behenate, mineral oil, sodium stearyl fumarate, stearic acid, hydrogenated vegetable oil and talc.

Glidants as used in the present invention comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants as used in the present invention comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the invention facilitates in the release of drug in the latter stage and thereby completely releasing the drug from the dosage form.

Suspending agents as used in the present invention comprises but not limited to gums like xanthan gum, guar gum; sorbitol; glycerol; polyvinyl alcohol; polyvinyl pyrrolidone; polyethylene oxide; cellulose derivatives, such as hydroxypropylmethylcellulose or a salt thereof, alkyl ether of cellulose, such as methylcellulose, ethylcellulose, hydroxyethylcellulose, hydroxyethylmethylcellose and mixtures thereof.

Examples of gums that may be used in the present invention comprise but not limited to xanthan gum, acacia, tragacanth as suspending agents, wehrein the most preferable gum is xanthan gum.

Buffering agent as used in the present invention comprises but not limited to monosodium citrate, sodium phopsphate, disodium phosphate, sodium acetate, wherein the most prefereble buffering agent is monosodium citrate.

Coloring agents as used in the present invention comprises but not limited to titanium dioxide, amaranth, tartarazine, wherein the most prefereble coloring agent is titanium dioxide.

Examples of sweetening agents and flavouring agents comprises but not limited to sugar alcohols, sugars, liquid glucose, sucrose, sachharine sodium, banana flavouring, vanilla flavouring, tutti frutty flavor, xylitol, sorbitol, mannitol, erythritol and the like.

Examples of sugar alcohols that may be used in the present invention include but not limited to sorbitol, erythritol, D-mannitol, sucrose and the like, wherein the most preferable sugar alcohol is sorbitol.

Examples of preservatives used in the invention comprises but are not limited to, sodium benzoate, chlorhexidine; methyl paraben; propyl paraben; butyl paraben and their salts; diazolidinyl urea; quaternary compounds like benzalkonium chloride and cetylpyridinium chloride, phenyl ethyl alcohol and the like. More preferably, compositions of the invention comprises sodium benzoate as preservative.

In another embodiment, the present invention provides a pharmaceutical composition comprising two different populations: i) first population comprising oseltamivir or a pharmaceutically acceptable salt thereof and sugar alcohol and ii) second population comprising sugar alcohol.

In another embodiment, a pharmaceutical composition comprising two different populations: i) first population comprising oseltamivir or a pharmaceutically acceptable salt thereof and sorbitol and ii) second population comprising sorbitol.

In another embodiment, a pharmaceutical composition comprising two different populations: i) first population is in the form of powder comprising oseltamivir or a pharmaceutically acceptable salt thereof and sugar alcohol and ii) second population is in the form of granules comprising sugar alcohol.

In another embodiment, a pharmaceutical composition comprising two different populations: i) first population is in the form of powder comprising oseltamivir or a pharmaceutically acceptable salt thereof and sorbitol and ii) second population is in the form of granules comprising sorbitol.

In another embodiment, a pharmaceutical composition comprising two different populations: i) first population comprising about 2-6% by weight of oseltamivir or a pharmaceutical acceptable salt thereof; 5-95% by weight of a sweetening agent and 0.2-5% by weight of a suspending agent; and ii) a second population comprising about 30-90% by weight of a first sweetening agent, about 1-10% by weight of buffer, about 0.01-0.50% by weight of a second sweetening agent, and about 0.20-5.0% by weight of a coloring agent, wherein second population does not contain oseltamivir or a pharmaceutically acceptable salt thereof.

In another embodiment, a pharmaceutical composition of invetion, the sweetening agent in the first population is selected from the group consisting of sugar alcohols like sorbitol, erythritol, D-mannitol; the suspending agent in the first population is selected from the group consisting of xanthan gum, polyvinyl alcohol, polyvinyl pyrrolidone, polyethylene oxide; the first sweetening agent in the second population is selected from the group consisting of sucrose, sachharine sodium, banana flavouring, vanilla flavouring, tutti frutty flavor, xylitol, sorbitol, mannitol, erythritol ; the buffer in the second population is selected from the group consisting of monosodium citrate, sodium phopsphate, disodium phosphate, sodium acetate; the second sweetening agent in the second population is selected from the group consisting of sucrose, sachharine sodium, banana flavouring, vanilla flavouring, tutti frutty flavor, xylitol, sorbitol, mannitol, erythritol; and the coloring agent in the second population is selected from the group consisting of titanium dioxide, amaranth, tartarazine.

In another embodiment, a pharmaceutical composition comprising two different populations: i) first population is in the form of powder comprising oseltamivir or a pharmaceutically acceptable salt thereof, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof.

In another embodiment, a pharmaceutical composition comprising two different populations: i) first population is in the form of powder comprising oseltamivir or a pharmaceutically acceptable salt thereof, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, and wherein the composition is stable throughout its shelf life.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, and wherein the composition is stable throughout its shelf life.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, wherein the composition is stable in real-time stability conditions as well as long-term stability conditions (30° C.±2° C./65% RH±5% RH).

According to US Pharmacopoeia monograph for Oseltamivir Phosphate capsules, three impurities are identified to be associated with Oseltamivir, they are Impurity A, B and C.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, wherein the composition has impurity A not more than 2.0% by weight as per the USP monograph for Oseltamivir Phosphate Capsules when kept in real-time and long-term stability conditions.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, wherein the composition has impurity B not more than 0.3% by weight as per the USP monograph for Oseltamivir Phosphate Capsules when kept in real-time and long-term stability conditions.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the second population does not contain oseltamivir or a pharmaceutically acceptable salt thereof, wherein the composition has impurity C not more than 0.5% by weight as per the USP monograph for Oseltamivir Phosphate Capsules, when kept in real-time and long-term stability conditions.

In another embodiment pharmaceutical composition of the invention has impurity A not more than 2.0% by weight, impurity B not more than 0.3% by weight and impurity C not more than 0.5% by weight as per the USP monograph for Oseltamivir Phosphate Capsules, while kept in real-time and long-term stability conditions.

In another embodiment, a pharmaceutical composition comprising two different populations: i) first population is in the form of granules comprising oseltamivir or a pharmaceutically acceptable salt thereof and sugar alcohol and ii) second population is in the form of granules comprising sugar alcohol.

In another embodiment, a pharmaceutical composition comprising two different populations: i) first population is in the form of granules comprising oseltamivir or a pharmaceutically acceptable salt thereof and sorbitol and ii) second population is in the form of granules comprising sorbitol.

In another embodiment, a pharmaceutical composition comprising two different populations: i) first population is in the form of granules comprising oseltamivir or a pharmaceutically acceptable salt thereof and sorbitol and ii) second population is in the form of granules comprising sorbitol and Titanium dioxide.

In another embodiment, a pharmaceutical composition comprising two different populations: i) first population is in the form of granules comprising oseltamivir or a pharmaceutically acceptable salt thereof and sorbitol and ii) second population is in the form of granules comprising sorbitol and Sodium benzoate.

In another embodiment, a pharmaceutical composition comprising two different populations: i) first population is in the form of granules comprising oseltamivir or a pharmaceutically acceptable salt thereof and sugar alcohol and ii) second population is in the form of powder comprising sugar alcohol.

In another embodiment, a pharmaceutical composition comprising two different populations: i) first population is in the form of granules comprising oseltamivir or a pharmaceutically acceptable salt thereof and sorbitol and ii) second population is in the form of powder comprising sorbitol.

In another embodiment, a process of preparing oseltamivir composition comprising filling two different populations into container using two different nozzles.

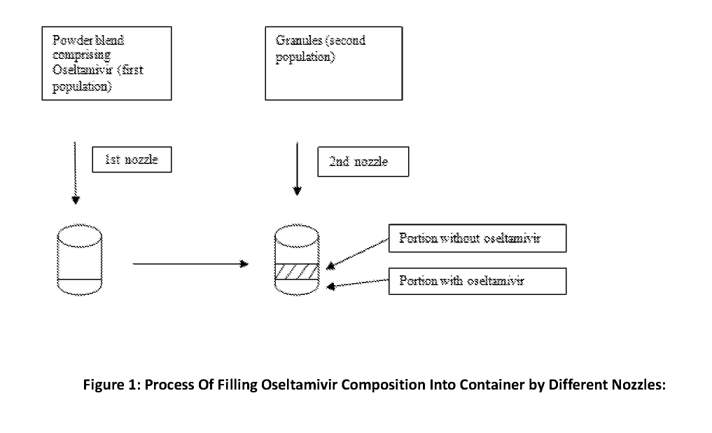

In another embodiment, a process of preparing oseltamivir composition comprising:
i. mixing oseltamivir or a pharmaceutically acceptable salt thereof with one or more pharmaceutically acceptable excipients to form first population;
ii. forming granules of pharmaceutically acceptable excipients by using granulation technique to form second population;
iii. filling first population of step (i) into the container with first nozzle;
iv. filling second population of step (ii) into the container of step (iii) by second nozzle as described in FIG. 1.

In another embodiment, a process of preparing oseltamivir composition comprising:
i. mixing oseltamivir or a pharmaceutically acceptable salt thereof with one or more pharmaceutically acceptable excipients to form first population;
ii. forming granules of pharmaceutically acceptable excipients by using granulation technique to form second population;
iii. filling first population of step (i) into the container with first nozzle;
iv. filling second population of step (ii) into the container of step (iii) by second nozzle as described in FIG. 1.

In another embodiment, a process of preparing oseltamivir composition comprising:
i. forming granules of oseltamivir or a pharmaceutically acceptable salt thereof with one or more pharmaceutically acceptable excipients to form first population;
ii. forming granules of pharmaceutically acceptable excipients by using granulation technique to form second population;
iii. filling first population of step (i) into the container with first nozzle;
iv. filling second population of step (ii) into the container of step (iii) by second nozzle.

In another embodiment, a process of preparing oseltamivir composition comprising:
i. forming granules of oseltamivir or a pharmaceutically acceptable salt thereof with one or more pharmaceutically acceptable excipients to form first population;
ii. mixing one or more pharmaceutically acceptable excipients to form second population;
iii. filling first population of step (i) into the container with first nozzle;
iv. filling second population of step (ii) into the container of step (iii) by second nozzle.

In another embodiment, a process of preparing pharmaceutical composition of invention comprise two different populations, wherein two populations are admixed together.

In another embodiment, a pharmaceutical composition comprising two different populations: i) first population is in the form of powder comprising oseltamivir phosphate, sorbitol and xanthan gum and ii) second population is in the form of granules comprising sorbitol, monosoidum citrate, saccharin sodium, sodium benzoate and titanium dioxide, wherein the composition is constitued into suspension using water as vehicle. Such suspension is stable throughout its shelf life.

In another embodiment, pharmaceutical compositions according to the present invention can be used for prevention or treatment of influenza virus infection and conditions associated with the infection selected from bronchitis, pneumonia, generalized pain and fever.

The following examples are illustrative of the present invention, and the examples should not be considered as limiting the scope of this invention in any way, as these examples and other equivalents thereof will become apparent to those versed in the art, in the light of the present disclosure, and the claims.

### EXAMPLES

### Example 1

| | % w/w |
|---|---|
| First Population | |
| 1. Oseltamivir Phosphate | 2-6 |
| 2. Sorbitol | 5-95 |
| 3. Xanthan Gum | 0.20-5.0 |
| Second Population | |
| 4. Sorbitol | 30-90 |
| 5. Monosodium Citrate | 1-10 |
| 6. Saccharin Sodium | 0.01-0.50 |
| 7. Sodium Benzoate | 0.050-0.50 |
| 8. Titanium Dioxide | 0.20-5.0 |
| 9. Dehydrated Alcohol | Q.S |
| 10. Purified Water | Q.S |
| Extragranular | |
| 11. Flavor | 0.20-5.0 |

Procedure:
i. Mix Oseltamivir Phosphate with Sorbitol and Xanthan gum to form powder blend.
ii. Prepare Sodium Benzoate granules by granulation using water and alcohol.
iii. Dry the granules.
iv. Blend dried granules of step (ii) with flavor.
v. Fill powder blend of step (i) and granules of step (iii) sequentially into container or suitable primary pack as described in FIG. 1.

### Example 2

| | % w/w |
|---|---|
| First Population | |
| 1. Oseltamivir Phosphate | 3.97 |
| 2. Sorbitol | 34.53 |
| 3. Xanthan Gum | 1.50 |
| Second Population | |
| 4. Sorbitol | 51.15 |
| 5. Monosodium Citrate | 5.51 |
| 6. Saccharin Sodium | 0.10 |
| 7. Sodium Benzoate | 0.25 |
| 8. Titanium Dioxide | 1.50 |
| 9. Dehydrated Alcohol | Q.S |
| 10. Purified Water | Q.S |
| Extragranular | |
| 11. Flavor | 1.50 |

Procedure:
i. Mix Oseltamivir Phosphate with Sorbitol and Xanthan gum to form powder blend.
ii. Prepare Sodium Benzoate granules by granulation using water and alcohol.
iii. Blend dried granules of step (ii) with flavor.
iv. Fill powder blend of step (i) and granules of step (iii) sequentially into container or suitable primary pack as described in FIG. 1.

### Example 3

| | % w/w |
|---|---|
| Oseltamivir Phosphate | |
| Granules | |
| 1. Oseltamivir Phosphate | 3.96 |
| 2. Sorbitol | 42.85 |
| 3. Monosodium Citrate | 2.75 |
| 4. Saccharin Sodium | 0.10 |
| 5. Sodium Benzoate | 0.25 |
| 6. Water | Q.S |
| 7. Ethanol | Q.S |
| Titanium Dioxide Granules | |
| 8. Sorbitol | 42.85 |
| 9. Monosodium Citrate | 2.75 |
| 10. Titanium Dioxide | 1.50 |
| 11. Dehydrated Alcohol | Q.S |
| Extragranular Portion | |
| 12. Flavor | 1.50 |
| 13. Xanthan Gum | 1.50 |

Procedure:
A. Oseltamivir Phosphate Granules
i. Mix Oseltamivir Phosphate with Sorbitol and Monosodium Citrate
ii. Dissolve sodium benzoate and saccharine sodium in solvent mixture.
iii. Granulate mixture of step (i) using solution of step (ii).
B. Titanium Dioxide Granules
iv. Mix Sorbitol, Monosodium Citrate and Titanium Dioxide.
v. Granulate mixture of step (iv) using alcohol to form Titanium Dioxide Granules.
C. Extragranular Portion
vi. Both Oseltamivir Phosphate granules and Titanium dioxide granules mixed along with flavor and Xanthan gum to form final blend.
vii. This blend was poured into bottle or suitable primary pack as a single blend.

### Example 4

| | % w/w |
|---|---|
| Oseltamivir Phosphate Granules | |
| 1. Oseltamivir Phosphate | 2-6 |
| 2. Sorbitol | 20-70 |
| 3. Monosodium Citrate | 0-10 |
| 4. Saccharin Sodium | 0.01-0.50 |
| 5. Sodium Benzoate | 0.05-0.50 |
| 6. Water | Q.S |
| 7. Ethanol | Q.S |
| Titanium Dioxide Granules | |
| 8. Sorbitol | 20-70 |
| 9. Monosodium Citrate | 0-10 |
| 10. Titanium Dioxide | 0.2-5.0 |
| 11. Dehydrated Alcohol | Q.S |
| Extragranular Portion | |
| 12. Flavor | 0.20-5.0 |
| 13. Xanthan Gum | 0.20-5.0 |

Procedure:
Oseltamivir Phosphate Granules
i. Mix Oseltamivir Phosphate with Sorbitol and Monosodium Citrate
ii. Dissolve sodium benzoate and saccharine sodium in solvent mixture.
iii. Granulate mixture of step (i) using solution of step (ii).
Titanium Dioxide Granules
iv. Mix Sorbitol, Monosodium Citrate and Titanium Dioxide.
v. Granulate mixture of step (iv) using alcohol to form Titanium Dioxide Granules.
Extragranular Portion
vi. Both Oseltamivir Phosphate granules and Titanium dioxide granules mixed along with flavor and Xanthan gum to form final blend.
vii. Pour blend of step (vi) into bottle or suitable primary pack as a single blend.

### Example 5

| | % w/w |
|---|---|
| Oseltamivir Phosphate Granules | |
| 1. Oseltamivir Phosphate | 3.96 |
| 2. Sorbitol | 25.71 |
| 3. Monosodium Citrate | 5.50 |
| 4. Saccharin Sodium | 0.10 |
| 5. Sodium Benzoate | 0.25 |
| 6. Water | — |
| 7. Ethanol | — |
| Titanium Dioxide Granules | |
| 8. Sorbitol | 59.99 |
| 10. Titanium Dioxide | 1.50 |
| 11. Dehydrated Alcohol | — |
| Extragranular Portion | |
| 12. Flavor | 1.50 |
| 13. Xanthan Gum | 1.50 |

Procedure:
Oseltamivir Phosphate Granules
i. Mix Oseltamivir Phosphate with Sorbitol and Monosodium Citrate
ii. Dissolve sodium benzoate and saccharine sodium in solvent mixture.
iii. Granulate mixture of step (i) using solution of step (ii).
Titanium Dioxide Granules
iv. Suspend Titanium Dioxide in Ethanol.
v. Granulate Sorbitol using suspension of step (iv) to form Titanium Dioxide Granules.
Extragranular Portion
vi. Both Sorbitol and Titanium Dioxide granules mixed along with extragranular material like flavor and Xanthan gum to form final blend.
vii. Pour blend of step (vi) into bottle or suitable primary pack as a single blend.

### Example 6

| | % w/w |
|---|---|
| Oseltamivir Phosphate Granules | |
| 1. Oseltamivir Phosphate | 3.97 |
| 2. Sorbitol | 42.68 |
| 3. Dehydrated Alcohol | — |
| Sodium Benzoate Granules | |
| 4. Sorbitol | 43.0 |
| 5. Saccharin Sodium | 0.10 |
| 6. Sodium Benzoate | 0.25 |
| 7. Dehydrated Alcohol | — |
| 8. Purified Water | — |
| Extragranular Excipients | |
| 9. Flavor | 1.50 |
| 10. Xanthan Gum | 1.0 |
| 11. Titanium Dioxide | 2.0 |
| 12. Monosodium Citrate | 5.50 |

Procedure:
i. Mix Oseltamivir Phosphate with Sorbitol.
ii. Granulate mixture of step (i) using alcohol.
iii. Dissolve Sodium Benzoate and Saccharine Sodium is in ethanol: water mixture.
iv. Adsorb solvent mixture of step (iii) over Sorbitol to form granules.
v. Mix two granules of step (ii) and (iv) with Extragranular Excipients to form Final Blend.
vi. Pour blend of step (v) into bottle or suitable primary pack as a single blend.

TABLE 1
Stablity Data for Example 2 under Real-time stability condition:

| IMPURITIES | INITIAL | 6 MONTHS |
|---|---|---|
| Impurity A | 0.04 | 0.06 |
| Impurity B | 0.03 | 0.09 |
| Impurity C | 0.02 | 0.07 |

The real time stability study of Example 2 of invetion is carried our for the period of six months and the data for Impuritied A, B, and C is disclosed in Table 2 above.

1. A pharmaceutical composition comprising two different populations: i) a first population comprising oseltamivir or a pharmaceutical acceptable salt thereof and one or more pharmaceutically acceptable excipients and ii) a second population comprising one or more pharmaceutically acceptable excipients suitable for oseltamivir, wherein second population does not contain oseltamivir or a pharmaceutically acceptable salt thereof.

2. The pharmaceutical composition according to claim 1, wherein the first population is in the form of a powder and the second population is in the form of granules.

3. The pharmaceutical composition according to claim 1, wherein the first population is in the form of granules and the second population is in the form of granules.

4. The pharmaceutical composition according to claim 1, wherein the first population is in the form of granules and the second population is in the form of a powder.

5. The pharmaceutical composition according to claim 1, wherein the ratio of first population to the second population is in the range of 1:99 to 99:1 by weight based on the total weight of the composition.

6. The pharmaceutical composition according to claim 1, wherein the first population comprises oseltamivir or a pharmaceutically acceptable salt thereof in an amount of about 5% to about 80% by weight based on the total weight of first population.

7. The pharmaceutical composition according to claim 1, wherein the composition comprises a mixture of granules and powder for constitution into suspension.

8. A pharmaceutical composition comprising as of claim 1, wherein the first population comprising about 2-6% by weight of oseltamivir or a pharmaceutical acceptable salt thereof; 5-95% by weight of a sweetening agent and 0.2-5% by weight of a suspending agent; and the second population comprising about 30-90% by weight of a first sweetening agent, about 1-10% by weight of buffer, about 0.01-0.50% by weight of a second sweetening agent, and about 0.20-5.0% by weight of a coloring agent,

9. The pharmaceutical composition of claim 8, wherein the sweetening agent in the first population is selected from the group consisting of sugar alcohols like sorbitol, erythritol, D-mannitol; the suspending agent in the first population is selected from the group consisting of xanthan gum, polyvinyl alcohol, polyvinyl pyrrolidone, polyethylene oxide; the first sweetening agent in the second population is selected from the group consisting of sucrose, sachharine sodium, banana flavouring, vanilla flavouring, tutti frutty flavor, xylitol, sorbitol, mannitol, erythritol-; the buffer in the second population is selected from the group consisting of monosodium citrate, sodium phopsphate, disodium phosphate, sodium acetate; the second sweetening agent in the second population is selected from the group consisting of sucrose, sachharine sodium, banana flavouring, vanilla flavouring, tutti frutty flavor, xylitol, sorbitol, mannitol, erythritol; and the coloring agent in the second population is selected from the group consisting of titanium dioxide, amaranth, tartarazine.

10. A process for preparing an oseltamivir composition that is a suspension, comprising adding a liquid vehicle to the composition of claim 1 to form the suspension.

11. A pharmaceutical composition comprising two different populations: i) first population comprising oseltamivir or a pharmaceutical acceptable salt thereof and one or more pharmaceutically acceptable excipients and ii) second population comprising one or more pharmaceutically acceptable excipients, wherein second population does not contain oseltamivir or a pharmaceutically acceptable salt thereof,wherein the composition has impurity A not more than 2.0% by weight, impurity B not more than 0.3% by weight and impurity C not more than 0.5% by weight as per the USP monograph for Oseltamivir Phosphate Capsules, while kept in real-time stability conditions.

12. A process for preparing a composition as of claim 1, said process comprising the steps of:
i. mixing oseltamivir or a pharmaceutically acceptable salt thereof with one or more pharmaceutically acceptable excipients to form first population;
ii. forming granules of one or more pharmaceutically acceptable excipients by using a granulation technique to form a second population;
iii. filling the first population of step (i) into a container with a first nozzle; and
iv filling the second population of step (ii) into the container of step (iii) by a second nozzle.

13. The process of claim 12, further comprising adding a liquid vehicle after step iv to the container to form a suspension of the composition.
